(19) United States
(12) Patent Application Publication
(10) Pub. No.: US 2017/0172903 A1
(43) Pub. Date: Jun. 22, 2017

(54) COMPOSITION WITH OPTICAL EFFECTS

(71) Applicant: L'OREAL, Paris (FR)

(72) Inventors: Remi EL AKKARI, Kawasaki-shi, Kanagawa (JP); Anne-Laure BERNARD, New York, NY (US)

(21) Appl. No.: 15/128,589

(22) PCT Filed: Mar. 31, 2015
(86) PCT No.: PCT/JP2015/060686

§ 371 Date: Sep. 23, 2016

(30) Foreign Application Priority Data
Mar. 31, 2014 (JP) 2014-070941

(51) Int. Cl. A61K 8/92; A61Q 1/14; A61K 8/86; A61K 8/31; A61K 8/37; A61K 8/34

(52) U.S. Cl. CPC A61K 8/922; A61K 8/37; A61K 8/345; A61K 8/86; A61K 8/31; A61Q 1/14; A61K 2800/882; A61K 2800/262

(57) ABSTRACT

The present invention relates to a cosmetic composition, comprising: (a) at least one oil phase including at least one triglyceride oil; (b) at least one polyol phase including at least one polyol; and (c) at least one surfactant phase including at least one nonionic surfactant, wherein at least one of the phases (a), (b) and (c) is visually distinct from the other(s), and the amount of the triglyceride oil is 7.5% by weight or more, preferably 10.0% by weight or more, and more preferably 15.0% by weight or more, relative to the total weight of the composition. The cosmetic composition according to the present invention can be translucent and exhibit a warm color, in particular a shiny warm color, when mixed well, for example, when shaken by hand, and observed through normal white background light. These unique optical effects can stimulate potential consumers visually.

### TECHNICAL FIELD

The present invention relates to a composition which can be separated into a plurality of liquid phases which are visually distinct, in particular a cosmetic composition, as well as a cosmetic process using the same.

### BACKGROUND ART

Oils are commonly used in cosmetics in order to, for example, provide conditioning effects for skin or hair, or remove make-up.

For hair, oils are well known to provide softness and brightness. However, the feeling to touch provided by the application of oils onto the hair may not be preferable. Thus, hair cosmetics for conditioning the hair are often based on emulsions which include water and some conditioning agents such as oils, as well as, typically, cationic agents. Due to the anionic nature of the hair, some cationic agents are electrochemically adsorbed on the hair to provide softness to the hair. Also, hair cosmetics for deterging the hair include emulsions which include water and some surfactants which can solubilize sebum on the hair by incorporating it into micelles.

For skin, rinse-off skin care oils are often used for the body to provide moisture to the skin. However, they are often difficult to use because they are too fluid. In addition, the rinsability of the rinse-off skin care oils is not always sufficient. Furthermore, they are expensive. Also, cleansing oils are used to remove make-up and sebum. However, they have the same problems, because they are too fluid, they may be difficult to be rinsed off in some cases, and they are relatively expensive.

### DISCLOSURE OF INVENTION

In typical cosmetics for hair and skin, at least one oil as a conditioner is combined with at least one surfactant as an emulsifier or detergent, and with a polar phase such as water. This combination often results in an emulsion which has a classical aspect and texture such as white and creamy.

In the meanwhile, there is a need to stimulate potential consumers visually.

An objective of the present invention is to provide a composition, in particular a cosmetic composition, which can stimulate visually potential consumers while providing good cosmetic effects such as conditioning and cleansing effects, and can be easily rinsed off.

The above objective can be achieved by a composition comprising:

(a) at least one oil phase including at least one triglyceride oil;
(b) at least one polyol phase including at least one polyol; and
(c) at least one surfactant phase including at least one nonionic surfactant;
wherein
at least one of the phases (a), (b) and (c) is visually distinct from the other(s), and the amount of the triglyceride oil is 7.5% by weight or more, preferably 10.0% by weight or more, and more preferably 15.0% by weight or more, relative to the total weight of the composition.

The composition according to the present invention can be transparent or translucent and exhibit a warm color, in particular a shiny warm color, when mixed well, for example, when shaken by hand, and observed through normal white background light. These unique optical effects can stimulate potential consumers visually.

The composition according to the present invention can also impart a non-dry final feeling after the application thereof, while imparting a moisturized feeling.

The difference in the refractive indices of the (a) oil phase and the (b) polyol phase and/or the (a) oil phase and the (c) surfactant phase may be less than 0.0020, preferably less than 0.0015, and more preferably less than 0.0010.

The amount of the triglyceride oil may range 60.0% by weight or less, preferably 50.0% by weight or less, and more preferably 40.0% by weight or less, relative to the total weight of the composition.

The amount of the (a) oil phase may range from 20.0 to 70.0% by weight, preferably from 25.0 to 60.0% by weight, and more preferably from 30.0 to 50.0% by weight or less, relative to the total weight of the composition.

It is preferable that the polyol be selected from the group consisting of glycerin, diglycerin, polyglycerin, ethyleneglycol, diethyleneglycol, polyethyleneglycol, propyleneglycol, dipropyleneglycol, polypropyleneglycol, butyleneglycol, pentyleneglycol, hexyleneglycol, 1,3-propanediol, and 1,5-pentanediol.

The amount of the (b) polyol phase may range from 30.0 to 80.0% by weight, preferably from 40.0 to 70.0% by weight, and more preferably from 45.0 to 65.0% by weight, relative to the total weight of the composition.

It is preferable that the nonionic surfactant have an HLB value of 18.0 or less, preferably from 4.0 to 18.0, more preferably from 6.0 to 15.0, and even more preferably from 9.0 to 13.0.

It is preferable that the nonionic surfactant be selected from esters of polyols with fatty acids with a saturated or unsaturated chain containing, for example, from 8 to 24 carbon atoms, preferably 12 to 22 carbon atoms, and alkoxylated derivatives thereof, such as glyceryl esters of a C₈-C₂₄ fatty acid or acids and alkoxylated derivatives thereof, polyethylene glycol esters of a C₈-C₂₄ fatty acid or acids and alkoxylated derivatives thereof, sorbitol esters of a C₈-C₂₄ fatty acid or acids and alkoxylated derivatives thereof, sugar (sucrose, glucose, alkylglycose) esters of a C₈-C₂₄ fatty acid or acids and alkoxylated derivatives thereof, ethers of fatty alcohols, ethers of sugar and a C₈-C₂₄ fatty alcohol or alcohols, and mixtures thereof.

It is more preferable that the nonionic surfactant be selected from the group consisting of PEG-7 glyceryl cocoate, PEG-20 methylglucoside sesquistearate, PEG-20 glyceryl tri-isostearate, PG-5 dioleate, PG-4 diisostearate, PG-10 isostearate, PEG-8 isostearate, and PEG-60 hydrogenated castor oil.

The amount of the (c) surfactant phase may range from 0.1 to 20% by weight, preferably from 0.5 to 15% by weight, and more preferably from 1 to 10% by weight, relative to the total weight of the composition.

It is preferable that the (a) triglyceride oil be selected from the group consisting of: linseed oil, camellia oil, macadamia nut oil, sunflower oil, soybean oil, arara oil, corn oil, sasanqua oil, safflower oil, grapeseed oil, sesame oil, peanut oil, wheat germ oil, cottonseed oil, alfalfa oil, poppy oil, pumpkin oil, blackcurrant oil, evening primrose oil, millet oil, barley oil, quinoa oil, rye oil, candlenut oil, passion flower oil, musk rose oil, sweet almond oil, avocado oil, castor oil, olive oil, jojoba oil, groundnut oil, rapeseed oil, coconut oil, hazelnut oil, karite butter, shea butter, palm oil, apricot seed oil and calophyllumoil.

The composition according to the present invention may further comprise water in an amount of 2% by weight or less, preferably 1% by weight or less, and more preferably 0.5% by weight or less, relative to the total weight of the composition.

It is preferable that the composition according to the present invention be a cosmetic composition.

The present invention also relates to a cosmetic process for a dry or wet keratin substance comprising the step of applying the cosmetic composition according to the present invention to the keratin substance, with or without mixing the (a) oil phase, the (b) polyol phase and the (c) surfactant phase in the cosmetic composition before the step of applying the cosmetic composition to the keratin substance.

### BEST MODE FOR CARRYING OUT THE INVENTION

After diligent research, the inventors have discovered that it is possible to provide a composition, in particular a cosmetic composition, which can stimulate visually potential consumers while providing good cosmetic effects such as conditioning and cleansing effects, and can be easily rinsed off.

Thus, the composition according to the present invention comprises:

(a) at least one oil phase including at least one triglyceride oil;
(b) at least one polyol phase including at least one polyol; and
(c) at least one surfactant phase including at least one nonionic surfactant;
wherein
at least one of the phases (a), (b) and (c) is visually distinct from the other(s), and
the amount of the triglyceride oil is 7.5% by weight or more, preferably 10.0% by weight or more, and more preferably 15.0% by weight or more, relative to the total weight of the composition. The term “distinct” here means that the distinction of the different phases, in the composition according to the present invention, is possible visually, on a macroscopic scale, with the naked eyes. Thus, the separation between the different phrases in the composition according to the present invention can be seen with the naked eyes.

It is preferable that at least two of the three phases, and most preferably all three phases, i.e., the (a) oil phase, the (b) polyol phase, and the (c) surfactant phase in the composition according to the present invention be visually distinct after coming into a state of rest. In this case, the composition according to the present invention can have multiple liquid phases. Each liquid phase can provide any independent visual effect. Thus, the multi liquid phases can provide unique appearances to the composition according to the present invention. Each of the liquid phases can be transparent before the liquid phases are mixed.

On the other hand, the multiple liquid phases can disappear when they are mixed by, for example, shaking the composition according to the present invention to form a uniform phase. Thus, for example, if the refractive indexes of the multiple liquid phases are similar to each other, it is possible for the uniform phase to be translucent. The uniform phase can separate into the multiple liquid phases again by leaving it, without any shear force, for a certain period of time such as from a few minutes to several hours.

Hereafter, each of the phases constituting the composition according to the present invention will be described in a detailed manner.

### [Oil Phase]

The composition according to the present invention includes at least one oil phase including at least one triglyceride oil. Two or more triglyceride oils may be used in combination. Thus, a single type of triglyceride oil or a combination of different types of triglyceride oils may be used. The oil phase is in the form of a liquid at ambient temperature such as 25° C. under atmospheric pressure (760 mmHg or 10⁵ Pa).

The triglyceride oil comprises at least one fatty acid ester of glycerol, wherein the fatty acid may have different carbon chain length of, for example, from 4 to 30 carbon atoms, more preferably from 6 to 25 carbon atoms, and more preferably from 8 to 20 carbon atoms, and the carbon chain may be linear or branched, and saturated or unsaturated.

The triglyceride oil may be natural or synthetic triglyceride oil.

The natural triglyceride oil may preferably be selected from plant or animal oils.

The plant oil may be selected from the group consisting of: linseed oil, camellia oil, macadamia nut oil, sunflower oil, soybean oil, arara oil, corn oil, sasanqua oil, safflower oil, grapeseed oil, sesame oil, peanut oil, wheat germ oil, cottonseed oil, alfalfa oil, poppy oil, pumpkin oil, blackcurrant oil, evening primrose oil, millet oil, barley oil, quinoa oil, rye oil, candlenut oil, passion flower oil, musk rose oil, sweet almond oil, avocado oil, castor oil, olive oil, jojoba oil, groundnut oil, rapeseed oil, coconut oil, hazelnut oil, karite butter, shea butter, palm oil, apricot seed oil and calophyllumoil.

The animal oil may be selected from, for example, squalene, polyhydrosqualene, and squalane.

The synthetic triglyceride oil may be selected from, for example, caprylic/capric triglyceride, for instance those sold by the company Stéarineries Dubois or those sold under the names Miglyol® 810, 812 and 818 by the company Dynamit Nobel.

The amount of the triglyceride oil is 7.5% by weight or more, preferably 10.0% by weight or more, and more preferably 15.0% by weight or more, relative to the total weight of the composition.

The amount of the triglyceride oil may range 60.0% by weight or less, preferably 50.0% by weight or less, and more preferably 40.0% by weight or less, relative to the total weight of the composition.

The oil phase may also comprise at least one additional oil other than triglyceride oil. A single type of additional oil may be used, but two or more different types of additional oil may be used in combination.

Preferably, the additional oil used in the present invention does not contain a polyalkylenated or polyglycerolated group or a salified carboxylic group.

The additional oil may be selected from the group consisting of, for example, aliphatic hydrocarbons, esters of fatty alcohols and/or fatty acids other than animal or plant oils and synthetic triglycerides, fatty alcohols, fatty acids, and silicone oils. These additional oils may be volatile or non-volatile. It is preferable that the additional oil be selected from aliphatic hydrocarbons, esters of fatty alcohols and/or fatty acids other than animal or plant oils and synthetic triglycerides, and mixtures thereof.

As examples of aliphatic hydrocarbons, mention may be made of, for example, linear or branched hydrocarbons such as mineral oils (e.g., liquid paraffin) with a longer or shorter hydrocarbon chain or chains, paraffin, vaseline or petrolatum, naphthalenes, and the like;

hydrogenated polyisobutene such as Parleam, isoeicosan, polybutylene, polydecene, hydrogenated polydecene; decene/butene copolymers; and mixtures thereof.

As examples of other aliphatic hydrocarbons, mention may also be made of linear or branched, or possibly cyclic C₆-C₁₆ lower alkanes. Examples that may be mentioned include hexane, undecane, dodecane, tridecane and isoparaffins such as isohexadecane and isodecane.

As examples of the esters of fatty alcohols and/or of fatty acids, which are advantageously different from the animal or plant oils as well as the synthetic glycerides mentioned above, mention may be made especially of esters of saturated or unsaturated, linear or branched C₁-C₂₆ aliphatic mono- or polyacids and of saturated or unsaturated, linear or branched C₁-C₂₆ aliphatic mono- or polyalcohols, the total carbon number of the esters being greater than or equal to 10.

Among the monoesters, mention may be made of dihydroabietyl behenate; octyldodecyl behenate; isocetyl behenate; cetyl lactate; C₁₂-C₁₅ alkyl lactate; isostearyl lactate; lauryl lactate; linoleyl lactate; oleyl lactate; (iso)stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methylacetyl ricinoleate; myristyl stearate; octyl isononanoate; 2-ethylhexyl isononate; octyl palmitate; octyl pelargonate; octyl stearate; octyldodecyl erucate; oleyl erucate; ethyl and isopropyl palmitates; 2-ethylhexyl palmitate; 2-octyldecyl palmitate; alkyl myristates such as isopropyl, butyl, cetyl, 2-octyldodecyl, myristyl or stearyl myristate; hexyl stearate; butyl stearate; isobutyl stearate; dioctyl malate; hexyl laurate; and 2-hexyldecyl laurate.

Still within the context of this variant, esters of C₄-C₂₂ dicarboxylic or tricarboxylic acids and of C₁-C₂₂ alcohols and esters of mono-, di- or tricarboxylic acids and of C₂-C₂₆ di-, tri-, tetra- or pentahydroxy alcohols may also be used.

The following may especially be mentioned: diethyl sebacate; diisopropyl sebacate; diisopropyl adipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate; tridecyl erucate; triisopropyl citrate; triisostearyl citrate; glyceryl trilactate; glyceryl trioctanoate; trioctyldodecyl citrate; trioleyl citrate; propylene glycol dioctanoate; neopentyl glycol diheptanoate; diethylene glycol diisononanoate; and polyethylene glycol distearates.

Among the esters mentioned above, it is preferable to use ethyl, isopropyl, myristyl, cetyl or stearyl palmitate, 2-ethylhexyl palmitate, 2-octyldecyl palmitate, alkyl myristates such as isopropyl, butyl, cetyl or 2-octyldodecyl myristate, hexyl stearate, butyl stearate, isobutyl stearate, dioctyl malate, hexyl laurate, 2-hexyldecyl laurate, isononyl isononanoate or cetyl octanoate.

The oil phase may also comprise, as fatty esters, sugar esters and diesters of C₆-C₃₀ and preferably C₁₂-C₂₂ fatty acids. It is recalled that the term “sugar” means oxygen-bearing hydrocarbon-based compounds containing several alcohol functions, with or without aldehyde or ketone functions, and which contain at least 4 carbon atoms. These sugars may be monosaccharides, oligosaccharides or polysaccharides.

Examples of suitable sugars that may be mentioned include sucrose (or saccharose), glucose, galactose, ribose, fructose, maltose, mannose, arabinose, xylose and lactose, and derivatives thereof, especially alkyl derivatives, such as methyl derivatives, for instance methylglucose.

The sugar esters of fatty acids may be chosen especially from the group comprising the esters or mixtures of esters of sugars described previously and of linear or branched, saturated or unsaturated C₆-C₃₀ and preferably C₁₂-C₂₂ fatty acids. If they are unsaturated, these compounds may comprise one to three conjugated or non-conjugated carbon-carbon double bonds.

The esters according to this variant may also be chosen from mono-, di-, tri-, tetraesters and polyesters, and mixtures thereof.

These esters may be chosen, for example, from oleates, laurates, palmitates, myristates, behenates, cocoates, stearates, linoleates, linolenates, caprates and arachidonates, or mixtures thereof such as, especially, oleo-palmitate, oleo-stearate and palmito-stearate mixed esters.

It is more particularly preferable to use monoesters and diesters and especially sucrose, glucose or methylglucose mono- or dioleates, stearates, behenates, oleopalmitates, linoleates, linolenates and oleostearates.

An example that may be mentioned is the product sold under the name Glucate® DO by the company Amerchol, which is a methylglucose dioleate.

Examples of esters or mixtures of esters of sugar and of fatty acid that may also be mentioned include:
the products sold under the names F160, F140, F110, F90, F70 and SL40 by the company Crodesta, respectively denoting sucrose palmitostearates formed from 73% monoester and 27% diester and triester, from 61% monoester and 39% diester, triester and tetraester, from 52% monoester and 48% diester, triester and tetraester, from 45% monoester and 55% diester, triester and tetraester, from 39% monoester and 61% diester, triester and tetraester, and sucrose monolaurate;
the products sold under the name Ryoto Sugar Esters, for example referenced B370 and corresponding to sucrose behenate formed from 20% monoester and 80% di-triester-polyester;
the sucrose mono-dipalmito-stearate sold by the company Goldschmidt under the name Tegosoft® PSE.

The oil phase may include at least one fatty alcohol, and two or more fatty alcohols may be used.

The term “fatty alcohol” here means any saturated or unsaturated, linear or branched C₈-C₃₀ fatty alcohol, which is optionally substituted, in particular with one or more hydroxyl groups (in particular 1 to 4). If they are unsaturated, these compounds may comprise one to three conjugated or non-conjugated carbon-carbon double bonds. Preferably fatty alcohols are unsaturated and/or branched.

Among the C₈-C₃₀ fatty alcohols, C₁₂-C₂₂ fatty alcohols, for example, are used. Mention may be made among these of, isostearyl alcohol, oleyl alcohol, linoleyl alcohol, undecylenyl alcohol, palmitoleyl alcohol, linolenyl alcohol, erucyl alcohol, and mixtures thereof. In one embodiment, cetyl alcohol, stearyl alcohol or a mixture thereof (e.g., cetearyl alcohol), as well as myristyl alcohol, can be used as a solid fatty material. In one embodiment, isostearyl alcohol can be used as a liquid fatty material.

The oil phase may include at least one wax. Here, “wax” means a fatty compound substantially in the form of a solid at room temperature (25° C.) under atmospheric pressure (760 mmHg), and has a melting point generally of 35° C. or more. As the waxy fatty material, waxes generally used in cosmetics can be used alone or in combination thereof.

The wax may be a fatty alcohol.

In one embodiment, cetyl alcohol, stearyl alcohol or a mixture thereof (e.g., cetearyl alcohol), as well as myristyl alcohol, can be used as the solid fatty material.

The wax may also be chosen from carnauba wax, microcrystalline waxes, ozokerites, hydrogenated jojoba oil, polyethylene waxes such as the wax sold under the name “Performalene 400 Polyethylene” by the company New Phase Technologies, silicone waxes, for instance poly(C₂₄-C₂₈)alkylmethyldimethylsiloxane, such as the product sold under the name “Abil Wax 9810” by the company Goldschmidt, palm butter, the C₂₀-C₄₀ alkyl stearate sold under the name “Kester Wax K82H” by the company Kester Keunen, stearyl benzoate, shellac wax, and mixtures thereof. For example, a wax chosen from carnauba wax, candelilla wax, ozokerites, hydrogenated jojoba oil and polyethylene waxes is used. In at least one embodiment, the wax is preferably chosen from candelilla wax and ozokerite, and mixtures thereof.

The oil phase may include at least one fatty acid. Two or more fatty acids may be used. The fatty acids may be in acidic form (i.e., unsalified, to avoid soaps), and may be saturated or unsaturated and contain from 6 to 30 carbon atoms and in particular from 9 to 30 carbon atoms, which are optionally substituted, in particular with one or more hydroxyl groups (in particular 1 to 4). If they are unsaturated, these compounds may comprise one to three conjugated or non-conjugated carbon-carbon double bonds. They are more particularly chosen from myristic acid, palmitic acid, stearic acid, behenic acid, oleic acid, linoleic acid, linolenic acid and isostearic acid. Preferably the additional oil is not a fatty acid.

As examples of silicone oils, mention may be made of, for example, linear organopolysiloxanes such as dimethylpolysiloxane, methylphenylpolysiloxane, methylhydrogenpolysiloxane, and the like; cyclic organopolysiloxanes such as octamethylcyclotetrasiloxane, decamethylcyclopentasiloxane, dodecamethylcyclohexasiloxane, and the like; and mixtures thereof.

The oil phase may include at least one colorant, but it is possible that the oil phase be colorless.

It may be preferable that the oil phase is free from silicone(s). The term “free from” here means that the oil phase may contain only a small amount of silicone(s), and preferably no silicone(s). Thus, the amount of silicone(s) may be 5% by weight or less, preferably 3% by weight or less, and more preferably 1% by weight or less of silicone(s). It is particularly preferable that the oil phase contains no silicone(s).

The oil phase may include at least one lipophilic compound such as oil-soluble organic or inorganic compounds (e.g., some types of amino acids). The amount of the lipophilic compound is 50% by weight or less, relative to the total weight of the oil phase.

Thus, the amount of the additional oil(s) in the oil phase may range, for example, from 1 to 40% by weight, preferably from 2 to 30% by weight, and more preferably from 3 to 20% by weight, relative to the total weight of the composition.

The amount of the (a) oil phase may range from 20.0 to 70.0% by weight, preferably from 25.0 to 60.0% by weight, and more preferably from 30.0 to 50.0% by weight or less, relative to the total weight of the composition.

### [Polyol Phase]

The composition according to the present invention includes at least one polyol phase including at least one polyol. Two or more polyols may be used in combination. Thus, a single type of polyol or a combination of different types of polyols may be used. The polyol phase is in the form of a liquid at ambient temperature such as 25° C. under atmospheric pressure (760 mmHg or 10⁵ Pa).

The term “polyol” here means an alcohol having two or more hydroxy groups, and does not encompass a saccharide or a derivative thereof. The derivative of a saccharide includes a sugar alcohol which is obtained by reducing one or more carbonyl groups of a saccharide, as well as a saccharide or a sugar alcohol in which the hydrogen atom or atoms in one or more hydroxy groups thereof has or have been replaced with at least one substituent such as an alkyl group, a hydroxyalkyl group, an alkoxy group, an acylgroup or a carbonyl group.

Polyols used in the present invention are liquid at ambient temperature such as 25° C. under atmospheric pressure (760 mmHg or 10⁵ Pa).

The polyol may be a C₂₋₂₄ polyol, preferably a C₂₋₉ polyol, comprising at least 2 hydroxy groups, and preferably 2 to 5 hydroxy groups.

The polyol may be a natural or synthetic polyol. The polyol may have a linear, branched or cyclic molecular structure.

The polyol may be selected from glycerins and derivatives thereof, and glycols and derivatives thereof. The polyol may be selected from the group consisting of glycerin, diglycerin, polyglycerin, ethyleneglycol, diethyleneglycol, propyleneglycol, dipropyleneglycol, butyleneglycol, pentyleneglycol, hexyleneglycol, C₆-C₂₄ polyethyleneglycol, 1,3-propanediol, 1,4-butanediol, and 1,5-pentanediol.

The polyol phase may include at least one colorant, but it is possible that the polyol phase be colorless.

The polyol phase may include at least one compound which is soluble in the polyol phase such as some types of amino acids, nonionic active ingredients such as Vitamin C. The amount of the polyol-soluble compound may be 50% by weight or less relative to the total weight of the polyol phase.

The amount of the (b) polyol phase may range from 30.0 to 80.0% by weight, preferably from 40.0 to 70.0% by weight, and more preferably from 45.0 to 65.0% by weight, relative to the total weight of the composition.

### [Surfactant Phase]

The composition according to the present invention includes at least one surfactant phase including at least one nonionic surfactant. Two or more nonionic surfactants may be used in combination. Thus, a single type of nonionic surfactant or a combination of different types of nonionic surfactants may be used. The surfactant phase is in the form of a liquid at ambient temperature such as 25° C. under atmospheric pressure (760 mmHg or 10⁵ Pa).

### (Nonionic Surfactant)

The nonionic surfactants are compounds well known in themselves (see, e.g., in this regard, “Handbook of Surfactants” by M. R. Porter, Blackie & Son publishers (Glasgow and London 1991, pp. 116-178). Thus, they can, for example, be chosen from alcohols, alpha-diols, alkylphenols and esters of fatty acids, these compounds being ethoxylated, propoxylated or glycerolated and having at least one fatty chain comprising, for example, from 8 to 30 carbon atoms, it being possible for the number of ethylene oxide or propylene oxide groups to range from 2 to 50, and for the number of glycerol groups to range from 1 to 30. Maltose derivatives may also be mentioned. Non-limiting mention may also be made of copolymers of ethylene oxide and/or of propylene oxide; condensates of ethylene oxide and/or of propylene oxide with fatty alcohols; polyethoxylated fatty amides comprising, for example, from 2 to 30 mol of ethylene oxide; polyglycerolated fatty amides comprising, for example, from 1.5 to 5 glycerol groups, such as from 1.5 to 4; ethoxylated fatty acid esters of sorbitan comprising from 2 to 30 mol of ethylene oxide; ethoxylated oils of plant origin; fatty acid esters of sucrose; fatty acid esters of polyethylene glycol; polyethoxylated fatty acid mono or diesters of glycerol (C₆-C₂₄)alkylpolyglycosides; N—(C₆-C₂₄)alkylglucamine derivatives; amine oxides such as (C₁₀-C₁₄)alkylamine oxides or N—(C₁₀-C₁₄)acylaminopropylmorpholine oxides; and mixtures thereof.

Preferably, the nonionic surfactant may be a nonionic surfactant with an HLB of 18.0 or less, such as from 4.0 to 18.0, more preferably from 6.0 to 15.0 and furthermore preferably from 9.0 to 13.0. The HLB is the ratio between the hydrophilic part and the lipophilic part in the molecule. This term HLB is well known to those skilled in the art and is described in “The HLB system. A time-saving guide to emulsifier selection” (published by ICI Americas Inc., 1984).

The nonionic surfactants may preferably be chosen from polyoxyalkylenated or polyglycerolated nonionic surfactants. The oxyalkylene units are more particularly oxyethylene or oxypropylene units, or a combination thereof, and are preferably oxyethylene units.

Examples of oxyalkylenated nonionic surfactants that may be mentioned include: oxyalkylenated (C₈-C₂₄)alkylphenols,

saturated or unsaturated, linear or branched, oxyalkylenated C₈-C₃₀ alcohols,
saturated or unsaturated, linear or branched, oxyalkylenated C₈-C₃₀ amides,
esters of saturated or unsaturated, linear or branched, C₈-C₃₀ acids and of polyethylene glycols,
polyoxyalkylenated esters of saturated or unsaturated, linear or branched, C₈-C₃₀ acids and of sorbitol,
saturated or unsaturated, oxyalkylenated plant oils,
condensates of ethylene oxide and/or of propylene oxide, inter alia, alone or as mixtures.

The surfactants preferably contain a number of moles of ethylene oxide and/or of propylene oxide of between 2 and 100 and most preferably between 2 and 50. Advantageously, the nonionic surfactants do not comprise any oxypropylene units.

In accordance with one preferred embodiment of the invention, the oxyalkylenated nonionic surfactants are chosen from oxyethylenated C₈-C₃₀ alcohols.

Examples of ethoxylated fatty alcohols (or C₈-C₃₀ alcohols) that may be mentioned include the adducts of ethylene oxide with lauryl alcohol, especially those containing from 9 to 50 oxyethylene groups and more particularly those containing from 10 to 12 oxyethylene groups (Laureth-10 to Laureth-12, as the CTFA names); the adducts of ethylene oxide with behenyl alcohol, especially those containing from 9 to 50 oxyethylene groups (Beheneth-9 to Beheneth-50, as the CTFA names); the adducts of ethylene oxide with cetearyl alcohol (mixture of cetyl alcohol and stearyl alcohol), especially those containing from 10 to 30 oxyethylene groups (Ceteareth-10 to Ceteareth-30, as the CTFA names); the adducts of ethylene oxide with cetyl alcohol, especially those containing from 10 to 30 oxyethylene groups (Ceteth-10 to Ceteth-30, as the CTFA names); the adducts of ethylene oxide with stearyl alcohol, especially those containing from 10 to 30 oxyethylene groups (Steareth-10 to Steareth-30, as the CTFA names); the adducts of ethylene oxide with isostearyl alcohol, especially those containing from 10 to 50 oxyethylene groups (Isosteareth-10 to Isosteareth-50, as the CTFA names); and mixtures thereof.

As examples of polyglycerolated nonionic surfactants, polyglycerolated C₈-C₄₀ alcohols are preferably used.

In particular, the polyglycerolated C₈-C₄₀ alcohols correspond to the following formula:

RO—[CH₂—CH(CH₂OH)—O]ₘ—H or RO—[CH(CH₂OH)—CH₂O]ₘ—H

in which R represents a linear or branched C₈-C₄₀ and preferably C₈-C₃₀ alkyl or alkenyl radical, and m represents a number ranging from 1 to 30 and preferably from 1.5 to 10.

As examples of compounds that are suitable in the context of the invention, mention may be made of lauryl alcohol containing 4 mol of glycerol (INCI name: Polyglyceryl-4 Lauryl Ether), lauryl alcohol containing 1.5 mol of glycerol, oleyl alcohol containing 4 mol of glycerol (INCI name: Polyglyceryl-4 Oleyl Ether), oleyl alcohol containing 2 mol of glycerol (INCI name: Polyglyceryl-2 Oleyl Ether), cetearyl alcohol containing 2 mol of glycerol, cetearyl alcohol containing 6 mol of glycerol, oleocetyl alcohol containing 6 mol of glycerol, and octadecanol containing 6 mol of glycerol.

The alcohol may represent a mixture of alcohols in the same way that the value of m represents a statistical value, which means that, in a commercial product, several species of polyglycerolated fatty alcohol may coexist in the form of a mixture.

According to one of the embodiments according to the present invention, the nonionic surfactant may be selected from esters of polyols with fatty acids with a saturated or unsaturated chain containing for example from 8 to 24 carbon atoms, preferably 12 to 22 carbon atoms, and alkoxylated derivatives thereof, preferably with a number of alkyleneoxide of from 10 to 200, and more preferably from 10 to 100, such as glyceryl esters of a C₈-C₂₄, preferably C₁₂-C₂₂, fatty acid or acids and alkoxylated derivatives thereof, preferably with a number of alkyleneoxide of from 10 to 200, and more preferably from 10 to 100; polyethylene glycol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sorbitol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sugar (sucrose, glucose, alkylglycose) esters of a C₈-C₂₄, preferably C₁₂-C₂₂, fatty acid or acids and alkoxylated derivatives thereof, preferably with a number of alkyleneoxide of from 10 to 200, and more preferably from 10 to 100; ethers of fatty alcohols; ethers of sugar and a C₈-C₂₄, preferably C₁₂-C₂₂, fatty alcohol or alcohols; and mixtures thereof.

Examples of ethoxylated fatty esters that may be mentioned include the adducts of ethylene oxide with esters of lauric acid, palmitic acid, stearic acid or behenic acid, and mixtures thereof, especially those containing from 9 to 100 oxyethylene groups, such as PEG-9 to PEG-50 laurate (as the CTFA names: PEG-9 laurate to PEG-50 laurate); PEG-9 to PEG-50 palmitate (as the CTFA names: PEG-9 palmitate to PEG-50 palmitate); PEG-9 to PEG-50 stearate (as the CTFA names: PEG-9 stearate to PEG-50 stearate); PEG-9 to PEG-50 palmitostearate; PEG-9 to PEG-50 behenate (as the CTFA names: PEG-9 behenate to PEG-50 behenate); polyethylene glycol 100 EO monostearate (CTFA name: PEG-100 stearate); and mixtures thereof.

As glyceryl esters of fatty acids, glyceryl stearate (glyceryl mono-, di- and/or tristearate) (CTFA name: glyceryl stearate) or glyceryl ricinoleate and mixtures thereof can in particular be cited.

As glyceryl esters of C₈-C₂₄ alkoxylated fatty acids, polyethoxylated glyceryl stearate (glyceryl mono-, di- and/or tristearate) such as PEG-20 glyceryl stearate can for example be cited.

Mixtures of these surfactants, such as for example the product containing glyceryl stearate and PEG-100 stearate, marketed under the name ARLACEL 165 by Uniqema, and the product containing glyceryl stearate (glyceryl mono- and distearate) and potassium stearate marketed under the name TEGIN by Goldschmidt (CTFA name: glyceryl stearate SE), can also be used.

The sorbitol esters of C₈-C₂₄ fatty acids and alkoxylated derivatives thereof can be selected from sorbitan palmitate, sorbitan trioleate and esters of fatty acids and alkoxylated sorbitan containing for example from 20 to 100 EO, such as for example polyethylene sorbitan trioleate (polysorbate 85) or the compounds marketed under the trade names Tween 20 or Tween 60 by Ubiqema.

As esters of fatty acids and glucose or alkylglucose, in particular glucose palmitate, alkylglucose sesquistearates such as methylglucose sesquistearate, alkylglucose palmitates such as methylglucose or ethylglucose palmitate, methylglucoside fatty esters and more specifically the diester of methylglucoside and oleic acid (CTFA name: Methyl glucose dioleate), the mixed ester of methylglucoside and the mixture oleic acid/hydroxystearic acid (CTFA name: Methyl glucose dioleate/hydroxystearate), the ester of methylglucoside and isostearic acid (CTFA name: Methyl glucose isostearate), the ester of methylglucoside and lauric acid (CTFA name: Methyl glucose laurate), the mixture of monoester and diester of methylglucoside and isostearic acid (CTFA name: Methyl glucose sesqui-isostearate), the mixture of monoester and diester of methylglucoside and stearic acid (CTFA name: Methyl glucose sesquistearate) and in particular the product marketed under the name Glucate SS by AMERCHOL, and mixtures thereof can be cited.

As ethoxylated ethers of fatty acids and glucose or alkylglucose, ethoxylated ethers of fatty acids and methylglucose, and in particular the polyethylene glycol ether of the diester of methylglucose and stearic acid with about 20 moles of ethylene oxide (CTFA name: PEG-20 methyl glucose distearate) such as the product marketed under the name Glucam E-20 distearate by AMERCHOL, the polyethylene glycol ether of the mixture of monoester and diester of methyl-glucose and stearic acid with about 20 moles of ethylene oxide (CTFA name: PEG-20 methyl glucose sesquistearate) and in particular the product marketed under the name Glucamate SSE-20 by AMERCHOL and that marketed under the name Giillocose PSE-20 by GOLDSCHMIDT, and mixtures thereof, can for example be cited.

As sucrose esters, saccharose palmito-stearate, saccharose stearate and saccharose monolaurate can for example be cited.

As sugar ethers, alkylpolyglucosides can be used, and for example decylglucoside such as the product marketed under the name MYDOL 10 by Kao Chemicals, the product marketed under the name PLANTAREN 2000 by Henkel, and the product marketed under the name ORAMIX NS 10 by Seppic, caprylyl/capryl glucoside such as the product marketed under the name ORAMIX CG 110 by Seppic or under the name LUTENSOL GD 70 by BASF, laurylglucoside such as the products marketed under the names PLANTAREN 1200 N and PLANTACARE 1200 by Henkel, coco-glucoside such as the product marketed under the name PLANTACARE 818/UP by Henkel, cetostearyl glucoside possibly mixed with cetostearyl alcohol, marketed for example under the name MONTANOV 68 by Seppic, under the name TEGO-CARE CG90 by Goldschmidt and under the name EMULGADE KE3302 by Henkel, arachidyl glucoside, for example in the form of the mixture of arachidyl and behenyl alcohols and arachidyl glucoside marketed under the name MONTANOV 202 by Seppic, cocoylethylglucoside, for example in the form of the mixture (35/65) with cetyl and stearyl alcohols, marketed under the name MONTANOV 82 by Seppic, and mixtures thereof can in particular be cited.

Mixtures of glycerides of alkoxylated plant oils such as mixtures of ethoxylated (200 EO) palm and copra (7 EO) glycerides can also be cited.

It is preferable that the nonionic surfactant be selected from the group consisting of PEG-7 glyceryl cocoate, PEG-20 methylglucoside sesquistearate, PEG-20 glyceryl tri-isostearate, PG-5 dioleate, PG-4 diisostearate, PG-10 isostearate, PEG-8 isostearate, and PEG-60 hydrogenated castor oil.

Mixtures of these oxyethylenated derivatives of fatty alcohols and of fatty esters may also be used.

It is possible that the nonionic surfactant is soluble in the oil phase and/or the polyol phase.

The surfactant phase may also comprise at least one ionic surfactant. A single type of ionic surfactant may be used, but two or more different types of ionic surfactant may be used in combination. The ionic surfactant can be selected from cationic surfactants, anionic surfactants, and amphoteric surfactants.

### (Cationic Surfactant)

The cationic surfactant is not limited. The cationic surfactant may be selected from the group consisting of optionally polyoxyalkylenated, primary, secondary or tertiary fatty amine salts, quaternary ammonium salts, and mixtures thereof.

Examples of quaternary ammonium salts that may be mentioned include, but are not limited to:

those of general formula (I) below:

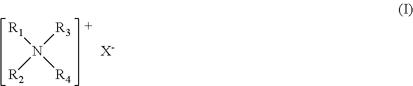

wherein
R₁, R₂, R₃, and R₄, which may be identical or different, are chosen from linear and branched aliphatic radicals comprising from 1 to 30 carbon atoms and optionally comprising heteroatoms such as oxygen, nitrogen, sulfur and halogens. The aliphatic radicals may be chosen, for example, from alkyl, alkoxy, C₂-C₆ polyoxyalkylene, alkylamide, (C₁₂-C₂₂)alkylamido(C₂-C₆)alkyl, (C₁₂-C₂₂)alkylacetate and hydroxyalkyl radicals; and aromatic radicals such as aryl and alkylaryl; and X⁻ is chosen from halides, phosphates, acetates, lactates, (C₂-C₆) alkyl sulfates and alkyl- or alkylaryl-sulfonates;
quaternary ammonium salts of imidazoline;
diquaternary ammonium salts; and
quaternary ammonium salts comprising at least one ester function.

Among the quaternary ammonium salts mentioned above that may be used in compositions according to the invention include, but are not limited to tetraalkylammonium chlorides, for instance dialkyldimethylammonium and alkyltrimethylammonium chlorides in which the alkyl radical comprises from about 12 to 22 carbon atoms, such as behenyltrimethylammonium, distearyldimethylammonium, cetyltrimethylammonium and benzyldimethylstearylammonium chloride; palmitylamidopropyltrimethylammonium chloride; and stearamidopropyldimethyl(myristyl acetate)ammonium chloride, sold under the name “Ceraphyl® 70” by the company Van Dyk.

According to one embodiment, the cationic surfactant that may be used in the compositions of the invention is chosen from quaternary ammonium salts, for example from behenyltrimethylammonium chloride, cetyltrimethylammonium chloride, Quaternium-83, Quaternium-87, Quaternium-22, behenylamidopropyl-2,3-dihydroxypropyldimethylammonium chloride, palmitylamidopropyltrimethylammonium chloride, and stearamidopropyldimethylamine.

### (Anionic Surfactant)

The anionic surfactant is not limited. The anionic surfactants may be chosen in particular from anionic derivatives of proteins of vegetable origin or of silk proteins, phosphates and alkyl phosphates, carboxylates, sulphosuccinates, amino acid derivatives, alkyl sulphates, alkyl ether sulphates, sulphonates, isethionates, taurates, alkyl sulphoacetates, polypeptides, anionic derivatives of alkyl polyglucosides, and their mixtures.

1) Anionic derivatives of proteins of vegetable origin are protein hydrolysates comprising a hydrophobic group, it being possible for the said hydrophobic group to be naturally present in the protein or to be added by reaction of the protein and/or of the protein hydrolysate with a hydrophobic compound. The proteins are of vegetable origin or derived from silk, and the hydrophobic group can in particular be a fatty chain, for example an alkyl chain comprising from 10 to 22 carbon atoms. Mention may more particularly be made, as anionic derivatives of proteins of vegetable origin, of apple, wheat, soybean or oat protein hydrolysates comprising an alkyl chain having from 10 to 22 carbon atoms, and their salts. The alkyl chain can in particular be a lauryl chain and the salt can be a sodium, potassium and/or ammonium salt.

Thus, mention may be made, as protein hydrolysates comprising a hydrophobic group, for example, of salts of protein hydrolysates where the protein is a silk protein modified by lauric acid, such as the product sold under the name Kawa Silk by Kawaken; salts of protein hydrolysates where the protein is a wheat protein modified by lauric acid, such as the potassium salt sold under the name Aminofoam W OR by Croda (CTFA name: potassium lauroyl wheat amino acids) and the sodium salt sold under the name Proteol LW 30 by Seppic (CTFA name: sodium lauroyl wheat amino acids); salts of protein hydrolysates where the protein is an oat protein comprising an alkyl chain having from 10 to 22 carbon atoms and more especially salts of protein hydrolysates where the protein is an oat protein modified by lauric acid, such as the sodium salt sold under the name Proteol OAT (30% aqueous solution) by Seppic (CTFA name: sodium lauroyl oat amino acids); or salts of apple protein hydrolysates comprising an alkyl chain having from 10 to 22 carbon atoms, such as the sodium salt sold under the name Proteol APL (30% aqueous/glycol solution) by Seppic (CTFA name: sodium cocoyl apple amino acids). Mention may also be made of the mixture of lauroyl amino acids (aspartic acid, glutamic acid, glycine, alanine) neutralized with sodium N-methylglycinate sold under the name Proteol SAV 50 S by Seppic (CTFA name: sodium cocoyl amino acids).

2) Mention may be made, as phosphates and alkyl phosphates, for example, of monoalkyl phosphates and dialkyl phosphates, such as lauryl monophosphate, sold under the name MAP 20® by Kao Chemicals, the potassium salt of dodecyl phosphate, the mixture of mono- and diesters (predominantly diester) sold under the name Crafol AP-31® by Cognis, the mixture of octyl phosphate monoester and diester, sold under the name Crafol AP-20® by Cognis, the mixture of ethoxylated (7 mol of EO) 2-butyloctyl phosphate monoester and diester, sold under the name Isofol 12 7 EO-Phosphate Ester® by Condea, the potassium or triethanolamine salt of mono(C₁₂-C₁₃)alkyl phosphate, sold under the references Arlatone MAP 230K-40® and Arlatone MAP 230T-60® by Uniqema, potassium lauryl phosphate, sold under the name Dermalcare MAP XC-99/09® by Rhodia Chimie, and potassium cetyl phosphate, sold under the name Arlatone MAP 160K by Uniqema.

3) Mention may be made, as carboxylates, of:
amido ether carboxylates (AEC), such as sodium lauryl amido ether carboxylate (3 EO), sold under the name Akypo Foam 30® by Kao Chemicals;
polyoxyethylenated carboxylic acid salts, such as oxyethylenated (6 EO) sodium lauryl ether carboxylate (65/25/10 C₁₂-C₁₄-C₁₆), sold under the name Akypo Soft 45 NV® by Kao Chemicals, polyoxyethylenated and carboxymethylated fatty acids originating from olive oil, sold under the name Olivem 400® by Biologia E Tecnologia, or oxyethylenated (6 EO) sodium tridecyl ether carboxylate, sold under the name Nikkol ECTD-6NEX® by Nikkol; and
salts of fatty acids (soaps) having a C₆ to C₂₂ alkyl chain which are neutralized with an organic or inorganic base, such as potassium hydroxide, sodium hydroxide, triethanolamine, N-methylglucamine, lysine and arginine.

4) Mention may in particular be made, as amino acid derivatives, of alkali salts of amino acids, such as:
sarcosinates, such as sodium lauroyl sarcosinate, sold under the name Sarkosyl NL 97® by Ciba or sold under the name Oramix L 30® by Seppic, sodium myristoyl sarcosinate, sold under the name Nikkol Sarcosinate MN® by Nikkol, or sodium palmitoyl sarcosinate, sold under the name Nikkol Sarcosinate PN® by Nikkol;
alaninates, such as sodium N-lauroyl-N-methylamidopropionate, sold under the name Sodium Nikkol Alaninate LN 30® by Nikkol or sold under the name Alanone ALE® by Kawaken, or triethanolamine N-lauroyl-N-methylalanine, sold under the name Alanone ALTA® by Kawaken;
glutamates, such as triethanolamine monococoyl glutamate, sold under the name Acylglutamate CT-12® by Ajinomoto, triethanolamine lauroyl glutamate, sold under the name Acylglutamate LT-12® by Ajinomoto;
aspartates, such as the mixture of triethanolamine N-lauroyl aspartate and triethanolamine N-myristoyl aspartate, sold under the name Asparack® by Mitsubishi;
glycine derivatives (glycinates), such as sodium N-cocoyl glycinate, sold under the names Amilite GCS-12® and Amilite GCK 12 by Ajinomoto;
citrates, such as the citric monoester of oxyethylenated (9 mol) coco alcohols, sold under the name Witconol EC 1129 by Goldschmidt; and
galacturonates, such as sodium dodecyl D-galactoside uronate, sold by Soliance.

5) Mention may be made, as sulphosuccinates, for example, of oxyethylenated (3 EO) lauryl (70/30 C₁₂/C₁₄) alcohol monosulphosuccinate, sold under the names Setacin 103 Special® and Rewopol SB-FA 30 K 4® by Witco, the disodium salt of a hemisulphosuccinate of C₁₂-C₁₄ alcohols, sold under the name Setacin F Special Paste® by Zschimmer Schwarz, oxyethylenated (2 EO) disodium oleamidosulphosuccinate, sold under the name Standapol SH 135® by Cognis, oxyethylenated (5 EO) lauramide monosulphosuccinate, sold under the name Lebon A-5000® by Sanyo, the disodium salt of oxyethylenated (10 EO) lauryl citrate monosulphosuccinate, sold under the name Rewopol SB CS 50® by Witco, or ricinoleic monoethanolamide monosulphosuccinate, sold under the name Rewoderm S 1333® by Witco. Use may also be made of polydimethylsiloxane sulphosuccinates, such as disodium PEG-12 dimethicone sulphosuccinate, sold under the name Mackanate-DC 30 by Maclntyre.

6) Mention may be made, as alkyl sulphates, for example, of triethanolamine lauryl sulphate (CTFA name: TEA lauryl sulphate), such as the product sold by Huntsman under the name Empicol TL40 FL or the product sold by Cognis under the name Texapon T42, which products are at 40% in aqueous solution. Mention may also be made of ammonium lauryl sulphate (CTFA name: ammonium lauryl sulphate), such as the product sold by Huntsman under the name Empicol AL 30FL, which is at 30% in aqueous solution.

7) Mention may be made, as alkyl ether sulphates, for example, of sodium lauryl ether sulphate (CTFA name: sodium laureth sulphate), such as that sold under the names Texapon N40 and Texapon AOS 225 UP by Cognis, or ammonium lauryl ether sulphate (CTFA name: ammonium laureth sulphate), such as that sold under the name Standapol EA-2 by Cognis.

8) Mention may be made, as sulphonates, for example, of α-olefinsulphonates, such as sodium α-olefinsulphonate (C₁₄-C₁₆), sold under the name Bio-Terge AS-40® by Stepan, sold under the names Witconate AOS Protégé® and Sulframine AOS PH 12® by Witco or sold under the name Bio-Terge AS-40 CG® by Stepan, secondary sodium olefinsulphonate, sold under the name Hostapur SAS 30® by Clariant; or linear alkylarylsulphonates, such as sodium xylenesulphonate, sold under the names Manrosol SXS30®, Manrosol SXS40® and Manrosol SXS93® by Manro.

9) Mention may be made, as isethionates, of acylisethionates, such as sodium cocoylisethionate, such as the product sold under the name Jordapon CI P® by Jordan.

10) Mention may be made, as taurates, of the sodium salt of palm kernel oil methyltaurate, sold under the name Hostapon CT Pate® by Clariant; N-acyl-N-methyltaurates, such as sodium N-cocoyl-N-methyltaurate, sold under the name Hostapon LT-SF® by Clariant or sold under the name Nikkol CMT-30-T® by Nikkol, Sodium Methyl Stearoyl Taurate sold under the name Nikkol SMT® or sodium palmitoyl methyltaurate, sold under the name Nikkol PMT® by Nikkol.

11) The anionic derivatives of alkyl polyglucosides can in particular be citrates, tartrates, sulphosuccinates, carbonates and glycerol ethers obtained from alkyl polyglucosides. Mention may be made, for example, of the sodium salt of cocoylpolyglucoside (1,4) tartaric ester, sold under the name Eucarol AGE-ET® by Cesalpinia, the disodium salt of cocoylpolyglucoside (1,4) sulphosuccinic ester, sold under the name Essai 512 MP® by Seppic, or the sodium salt of cocoylpolyglucoside (1,4) citric ester, sold under the name Eucarol AGE-EC® by Cesalpinia.

It is preferable that the amino acid derivatives be acyl glycine derivatives or glycine derivatives, in particular acyl glycine salt.

The acyl glycine derivatives or glycine derivatives can be chosen from acyl glycine salts (or acyl glycinates) or glycine salts (or glycinates), and in particular from the following.

i) Acyl glycinates of formula (I):

R—HNCH₂COOX (I)

in which
R represents an acyl group R′C═O, with R′, which represents a saturated or unsaturated, linear or branched, hydrocarbon chain, preferably comprising from 10 to 30 carbon atoms, preferably from 12 to 22 carbon atoms, more preferably from 14 to 22 carbon atoms, and better still from 16 to 20 carbon atoms, and
X represents a cation chosen, for example, from the ions of alkali metals, such as Na, Li or K, preferably Na or K, the ions of alkaline earth metals, such as Mg, ammonium groups and their mixtures.

The acyl group can in particular be chosen from the lauroyl, myristoyl, behenoyl, palmitoyl, stearoyl, isostearoyl, olivoyl, cocoyl or oleoyl groups and their mixtures.

Preferably, R is a cocoyl group.

ii) Glycinates of following formula (II):

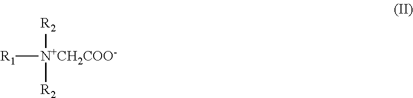

in which:
R₁ represents a saturated or unsaturated, linear or branched, hydrocarbon chain comprising from 10 to 30 carbon atoms, preferably from 12 to 22 carbon atoms and better still from 16 to 20 carbon atoms; R₁ is advantageously chosen from the lauryl, myristyl, palmityl, stearyl, cetyl, cetearyl or oleyl groups and their mixtures and preferably from the stearyl and oleyl groups,
the R₂ groups, which are identical or different, represent an R″OH group, R″ being an alkyl group comprising from 2 to 10 carbon atoms, preferably from 2 to 5 carbon atoms.

Mention may be made, as compounds of formula (I), for example, of the compounds carrying the INCI name sodium cocoyl glycinate, such as, for example, Amilite GCS-12, sold by Ajinomoto, or potassium cocoyl glycinate, such as, for example, Amilite GCK-12 from Ajinomoto.

Use may be made, as compounds of formula (II), of dihydroxyethyl oleyl glycinate or dihydroxyethyl stearyl glycinate.

### (Amphoteric Surfactant)

The amphoteric surfactant is not limited. The amphoteric or zwitterionic surfactants can be, for example (nonlimiting list), amine derivatives such as aliphatic secondary or tertiary amine, and optionally quaternized amine derivatives, in which the aliphatic radical is a linear or branched chain comprising 8 to 22 carbon atoms and containing at least one water-solubilizing anionic group (for example, carboxylate, sulphonate, sulphate, phosphate or phosphonate).

Among the amidoaminecarboxylated derivatives, mention may be made of the products sold under the name Miranol, as described in U.S. Pat. Nos. 2,528,378 and 2,781,354 and classified in the CTFA dictionary, 3rd edition, 1982 (the disclosures of which are incorporated herein by reference), under the names Amphocarboxyglycinates and Amphocarboxypropionates, with the respective structures:

R₁—CONHCH₂CH₂—N⁺(R₂)(R₃)(CH₂COO⁻)

in which:
R₁ denotes an alkyl radical of an acid R₁—COOH present in hydrolysed coconut oil, a heptyl, nonyl or undecyl radical,
R₂ denotes a beta-hydroxyethyl group, and
R₃ denotes a carboxymethyl group; and

R₁′—CONHCH₂CH₂—N(B)(C)

in which:
B represents —CH₂CH₂OX′,
C represents —(CH₂)_{z}—Y′, with z=1 or 2,
X′ denotes a —CH₂CH₂—COOH group, —CH₂—COOZ′, —CH₂CH₂—COOH, —CH₂CH₂—COOZ′ or a hydrogen atom,
Y′ denotes —COOH, —COOZ′, —CH₂—CHOH—SO₃Z′ or a —CH₂—CHOH—SO₃H radical,
Z′ represents an ion of an alkaline or alkaline earth metal such as sodium, an ammonium ion or an ion issued from an organic amine, and
R₁′ denotes an alkyl radical of an acid R₁′—COOH present in coconut oil or in hydrolysed linseed oil, an alkyl radical, such as a C₇, C₉, C₁₁ or C₁₃ alkyl radical, a C₁₇ alkyl radical and its iso form, or an unsaturated C₁₇ radical.

It is preferable that the amphoteric surfactant be selected from (C₈-C₂₄)-alkyl amphomonoacetates, (C₈-C₂₄)alkyl amphodiacetates, (C₈-C₂₄)alkyl amphomonopropionates, and (C₈-C₂₄)alkyl amphodipropionates

These compounds are classified in the CTFA dictionary, 5th edition, 1993, under the names Disodium Cocoamphodiacetate, Disodium Lauroamphodiacetate, Disodium Caprylamphodiacetate, Disodium Capryloamphodiacetate, Disodium Cocoamphodipropionate, Disodium Lauroamphopropionate, Disodium Caprylamphodipropionate, Disodium Caprylamphodipropionate, Lauroamphodipropionic acid and Cocoamphodipropionic acid.

By way of example, mention may be made of the cocoamphodiacetate sold under the trade name Miranol® C2M concentrate by the company Rhodia Chimie.

Preferably, the amphoteric surfactant may be a betaine.

The betaine-type amphoteric surfactant is preferably selected from the group consisting of alkylbetaines, alkylamidoalkylbetaines, alkylsulfobetaines, alkylphosphobetaines, and alkylamidoalkylsulfobetaines, in particular, (C₈-C₂₄)alkylbetaines, (C₈-C₂₄)alkylamido(C₁-C₈)alkylbetaines, (C₈-C₂₄)alkylsulphobetaines, and (C₈-C₂₄)alkylamido(C₁-C₈)alkylsulphobetaines. In one embodiment, the amphoteric surfactants of betaine type are chosen from (C₈-C₂₄)alkylbetaines, (C₈-C₂₄)alkylamido(C₁-C₈)alkylsulphobetaines, (C₈-C₂₄)alkylsulphobetaines, and alkyl(C₈-C₂₄)phosphobetaines.

Non-limiting examples that may be mentioned include the compounds classified in the CTFA dictionary, 9th edition, 2002, under the names cocobetaine, laurylbetaine, cetylbetaine, coco/oleamidopropylbetaine, cocamido propyl betaine, palmitamido propylbetaine, stearamidopropylbetaine, cocamidoethylbetaine, cocamidopropylhydroxysultaine, oleamidopropylhydroxysultaine, cocohydroxysultaine, laurylhydroxysultaine, and cocosultaine, alone or as mixtures.

The betaine-type amphoteric surfactant is preferably an alkylbetaine and an alkylamidoalkylbetaine, in particular cocobetaine and cocamidopropylbetaine.

The amount of the ionic surfactant(s) in the surfactant phase may range, for example, from 0.01 to 5% by weight, preferably from 0.1 to 4% by weight, and more preferably from 1 to 3% by weight, relative to the total weight of the composition.

The surfactant phase may include at least one colorant, but it is possible that the surfactant phase be colorless.

The amount of the (c) surfactant phase may range from 0.1 to 20% by weight, preferably from 0.5 to 15% by weight, and more preferably from 1 to 10% by weight, relative to the total weight of the composition.

### [Composition]

Preferably, the composition according to the present invention is substantially anhydrous. The term “substantially anhydrous” here means that the composition according to the present invention may contain only a small amount of water, and preferably no water. Thus, the amount of water may be 2% by weight or less, preferably 1% by weight or less, and more preferably 0.5% by weight or less relative to the total weight of the composition. It is particularly preferable that the composition according to the present invention contains no water as a distinct ingredient to be added intentionally. On the other hand, a small or trace amount of water may be present in the ingredient itself to be included in the composition according to the present invention.

According to the present invention, the (a) oil phase, the (b) polyol phase, and the (c) surfactant phase in the composition can directly contact each other. Typically, the above three phases may be packaged in a single container.

It is preferable that the difference in the refractive indices of the (a) oil phase and the (b) polyol phase and/or the (a) oil phase and the (c) surfactant phase be less than 0.0020, preferably less than 0.0015, and more preferably less than 0.0010. The small difference in refractive indices can make the composition according to the present invention translucent even when the three phases are mixed.

It is preferable that the (a) oil phase, the (b) polyol phase, and the (c) surfactant phase in the composition according to the present invention be capable of being spontaneously separated from each other. Preferably, at least two of the three phases are visually distinct after coming into a state of rest. More preferably, the three phases can be separated. This phase separation can visually stimulate the users of the composition according to the present invention. In one embodiment, the (c) surfactant phase in the composition according to the present invention can be present between the (a) oil phase and the (b) polyol phase. The phase separation of the composition according to the present invention can be caused by leaving it, without any shear force, for a certain period of time such as a few minutes to 24 hours, preferably 5 minutes to 24 hours, more preferably 10 minutes to 3 hours, even more preferably 10 minutes to 1 hour and 30 minutes.

It is preferable that the composition according to the present invention be capable of being forming a homogeneous phase when mixed by, for example, shaking the composition by hand. It is preferable that the homogeneous phase be in lamellar form, and viscous such that it does not drip off.

The viscosity of the composition according to the present invention is not particularly limited. The viscosity can be measured at 25° C. with viscosimeters or rheometers preferably with cone-plane geometry. Preferably, the viscosity of the composition according to the present invention can range, for example, from 1 to 2000 Pa·s, and preferably from 1 to 1000 Pa·s at 25° C. and 1 s⁻¹.

The composition according to the present invention may also comprise an effective amount of additives such as anionic, cationic, or amphoteric surfactants, thickeners, sequestering agents, UV screening agents, preserving agents, vitamins or provitamins, opacifiers, fragrances, plant extracts, humectants, waxes, fillers, colouring materials, antioxidants, proteins, and so on.

If any of the (a) oil phase, the (b) polyol phase, and the (c) surfactant phase is colored, the composition according to the present invention may be more visually attractive for users.

The composition according to the present invention can be prepared by mixing the above components by using a conventional mixing means such as a mixer and a homogenizer.

The composition according to the present invention can be used as, for example, a cosmetic composition for the skin or hair, such as hair rinse-off or leave on products (e.g., shampoos and conditioners), make-up removers (e.g., cleansing products), make-up products (e.g., foundations), body wash products, and skin moisturizing products.

Since the composition according to the present invention comprises not only oil(s) but also polyol(s), and nonionic surfactant(s), it can be prepared by using a less amount of oils as compared to conventional oil-based products, and therefore, this can reduce the cost to prepare the composition according to the present invention.

### [Cosmetic Process]

It is preferable that the composition according to the present invention be a cosmetic composition.

The cosmetic composition according to the present invention can be used in a cosmetic process for a keratin substance comprising the step of applying the cosmetic composition according to the present invention to a keratin substance. Keratin substance here means a material containing keratin as a main constituent element, and examples thereof include the skin, nail, lip, hair and the like.

The keratin substance can be in a dry state or in a wet state before application of the cosmetic composition according to the present invention. The application of the cosmetic composition according to the invention to the keratin substance may or may not be followed by rinsing the keratin substance. Before rinsing, the cosmetic composition according to the present invention can be left in contact with the keratin substance, for example, from 30 seconds to 30 minutes.

The cosmetic process according to the present invention for a dry or wet keratin substance comprises the step of applying the cosmetic composition according to the present invention to the keratin substance, with or without mixing the (a) oil phase, the (b) polyol phase and the (c) surfactant phase in the cosmetic composition before the step of applying the cosmetic composition to the keratin substance.

It is preferable that the cosmetic process for a keratin substance according to the present invention comprise the step of mixing the (a) oil phase, the (b) polyol phase and the (c) surfactant phase in the cosmetic composition, for example by hand, before the step of applying the cosmetic composition to the keratin substance.

By mixing the cosmetic composition according to the present invention, a homogeneous phase is formed. Since the homogeneous phase includes oil(s), polyol(s) and nonionic surfactant(s), the cosmetic effects provided by these ingredients can be provided uniformly to the keratin substance, which will result in good balance of cosmetic effects provided by each of the phases (a) to (c).

Since the homogeneous phase is not in the form of an O/W emulsion, oil and/or polyol (they are capable of forming a layer on a keratin substance which can inhibit the evaporation of water from the keratin substance, while they can solubilize hydrophobic or hydrophilic substances used in, for example, make-up cosmetics) can directly contact the keratin substance, and therefore, the cosmetic composition according to the present invention can provide superior moisturizing and/or cleansing effects.

Furthermore, due to the presence of the nonionic surfactant(s), the cosmetic composition according to the present invention can be easily rinsed off. In particular, when the cosmetic composition according to the present invention is used with water, it can easily form an emulsion, and can be smoothly removed from the keratin substance. The polyol can also be removed easily, when the cosmetic composition according to the present invention is used with water, because of its hydrophilic nature.

In addition, as compared to anionic, cationic or amphoteric surfactants, nonionic surfactants are less irritative, and therefore, the cosmetic composition according to the present invention can provide good feeling during use.

The cosmetic composition according to the present invention can be translucent even when the three phases are mixed, and can exhibit a warm color, in particular a shiny warm color when it is observed through normal white background light. These unique optical effects of the cosmetic composition according to the present invention can visually stimulate a user, for example give the user a warm feeling during use, in particular when the user looks up at the mixed cosmetic composition in a container through white light from a light source on the ceiling in, for example, a bath room.

### EXAMPLES

The present invention will be described in a more detailed manner by way of examples. However, these examples should not be construed as limiting the scope of the present invention.

### Example 1 and Comparative Example 1

### Preparation

The following compositions according to Example 1 (Ex. 1) and Comparative Example 1 (Comp. Ex. 1) were respectively prepared by mixing the ingredients shown in Table 1 at room temperature, and were poured into transparent vessels with the same volume. The numerical values for the amounts of the ingredients are all based on “% by weight” as active raw materials.

The compositions according to Example 1 and Comparative Example 1 showed the same aspect when they were left at rest. Each composition was separated into three phases, i.e., an oil phase, a surfactant phase and a polyol phase, in this order from the top to the bottom of the vessel. The sizes of the oil phase, the surfactant phase and the polyol phase in the composition according to Example 1 were almost the same as those of the composition according to Comparative Example 1. The oil phase and the surfactant phase were colored brown with the colorant shown in Table 1, while the polyol phase was colorless.

### [Refractive Indexes]

The refractive index of a phase composed of the ingredient(s) in Table 1 was calculated using the following formula:

$$n = \frac{{\sum }_{i = 1}^{p} {n}_{i} \frac{{c}_{i}}{{d}_{i}}}{{\sum }_{i = 1}^{p} \frac{{c}_{i}}{{d}_{i}}}$$

wherein
n is the refractive index of the whole phase;
nᵢ is the refractive index of the ingredient i;
cᵢ is the concentration in wt % of the ingredient i in the whole composition;
dᵢ is the density of the ingredient i; and
p is the number of ingredients.

The index of the oil and surfactant phases, the index of the polyol phase, and the difference between these indexes are shown in Table 1.

TABLE 1

| | | | | Comp. | |
|---|---|---|---|---|---|
| | | D | RI | Ex. 1 | Ex. 1 |
| Oil | Rapeseed Oil | 0.92 | 1.472 | 7 | 33.9 |
| | Ethylhexyl | 0.85 | 1.4453 | 35.8 | 8 |
| | Palmitate | | | | |
| Surfactant | PEG-20 Glyceryl | 1 | 1.4675 | 1.9994 | 1.9994 |
| | Triisostearate | | | | |
| Polyol | Glycerin | 1.26 | 1.472 | — | 14.1 |
| | PEG-8 | 1.13 | 1.466 | 24 | 42 |
| | Butylene Glycol | 1 | 1.439 | 31.2 | — |
| Colorant | Plant Extract | — | — | 0.0006 | 0.0006 |

| | | |
|---|---|---|
| Total | 100 | 100 |
| Refractive Index of Oil and Surfactant Phases | 1.4501 | 1.4666 |
| Refractive Index of Polyol Phase | 1.4499 | 1.4674 |
| Δ Index | 0.0002 | 0.0008 |

D: Density
RI: Refractive Index

### [Aspect After Shaking]

The translucency of the compositions according to Example 1 and Comparative Example 1 was evaluated by measuring the nephelometric turbidity of the compositions after being shaken by hand with a 2100Q Portable Turbidimeter (HACH) under the conditions that when the nephelometric turbidity of the composition was below 300 NTU, the composition was translucent.

The compositions according to Example 1 and Comparative Example 1 after being shaken were translucent.

### [Optical Properties]

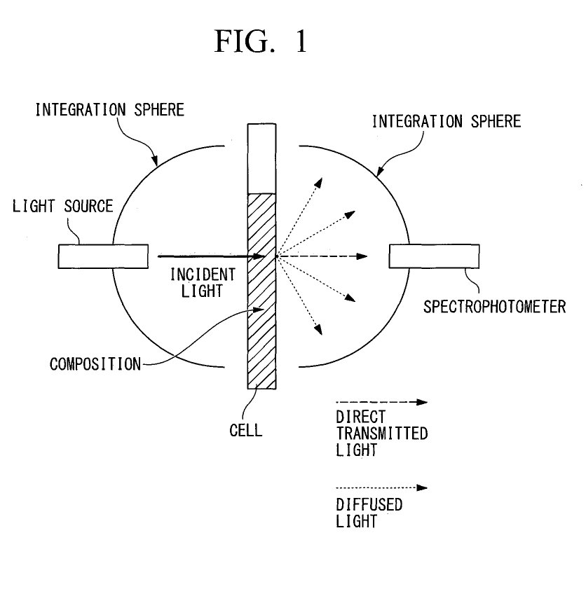

Each of the compositions according to Example 1 and Comparative Example 1 was shaken by hand, was immediately poured into a transparent cell with a thickness of 1 cm, and was immediately analyzed using a UV/VIS Spectrophotometer (JASCO V-550) within 1 minute as shown in FIG. 1 to measure the total transmittance and the scattered transmittance in the visible light region.

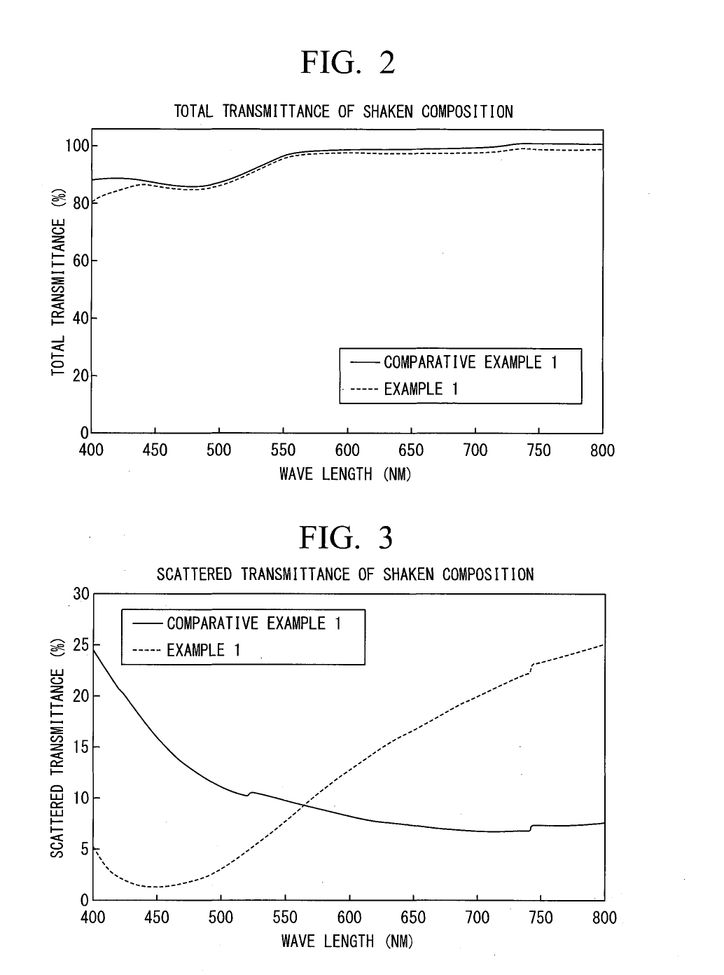

The total transmittance of each of the compositions according to Example 1 and Comparative Example 1 is shown in FIG. 2.

The total transmittance is the transmittance based on all the light which was transmitted through the above cell regardless of the direction of the transmitted light, relative to the incident light to the cell. In FIG. 1, all the light which transmitted through the above cell regardless of the direction of the transmitted light corresponds to the sum of the direct transmitted light and the diffused light.

As shown in FIG. 2, the total transmittances of the compositions according to Example 1 and Comparative Example 1 were close to 100%, and the transmittance curves thereof were very similar to each other. This means that the light absorptions of both compositions were similar to each other in the visible light region, and that both compositions have the same or similar color “under” normal visible white light.

The scattered transmittance of each of the compositions according to Example 1 and Comparative Example 1 is shown in FIG. 3.

The scattered transmittance is the transmittance based on all the scattered light which was transmitted through the above cell, relative to the incident light to the cell. All the scattered light here means all the light which was transmitted through the above cell regardless of the direction of the transmitted light subtracted by the light transmitted through the cell in line with the direction of the incident light. In FIG. 1, all the diffused light corresponds to the scattered light.

As shown in FIG. 3, the scattered transmittance curves of the compositions according to Example 1 and Comparative Example 1 were very different. It is clear that the composition according to Example 1 can scatter visible light with a longer wavelength, i.e., yellow-orange-red region, while the composition according to Comparative Example 1 can scatter visible light with a shorter wavelength, i.e., blue region. This means that if one looks at the composition according to Example 1 with a “background” of incident visible white light, the composition around the direct transmitted light looks yellow/orange/red, while if one looks at the composition according to Comparative Example 1 with the background of incident visible white light, the composition around the direct transmitted light had a pale white color.

Accordingly, the composition according to Example 1 with a larger amount of triglyceride oil (rapeseed oil) can provide a warm color “through” normal visible white background light.

It should be noted that a scattered transmittance of 15% or more provides shiny effects, in particular a shiny iridescent aspect. Therefore, the composition according to Example 1 which has a scattered transmittance of more than 15% in the region of 700 to 800 nm and less than 15% in the region of 400 to 500 nm can provide warm-color shiny effects, in particular a warm-color shiny iridescent aspect.

### Examples 2-9 and Comparative Examples 2-5

### Preparation

The following compositions according to Examples 2-9 (Ex. 2-9) and Comparative Examples 2-5 (Comp. Ex. 2-5) were respectively prepared by mixing the ingredients shown in Tables 2-3 at room temperature, and were poured into transparent vessels with the same volume. The numerical values for the amounts of the ingredients are all based on “% by weight” as active raw materials.

The compositions according to Examples 2-9 and Comparative Examples 2-5 showed the same aspect when they were left at rest. Each composition was separated into three phases, i.e., an oil phase, a surfactant phase and a polyol phase, in this order from the top to the bottom of the vessel. The sizes of the oil phase, the surfactant phase and the polyol phase in the compositions according to Examples 2-9 were almost the same as those of the compositions according to Comparative Examples 2-5. The oil phase, the surfactant phase and the polyol phases were colorless.

### [Refractive Indexes]

The refractive index of a phase composed of the ingredient(s) in Tables 2-3 was calculated as explained above. The index of the oil phase, the index of the polyol phase, and the difference between these indexes are shown in Tables 2-3.

### [Aspect After Shaking]

The compositions according to Examples 2-9 and Comparative Examples 2-5 were shaken by hand, and a visual evaluation was made regarding whether the composition was translucent or opaque. The results are shown in Tables 2-4.

### [Shiny Iridescent Aspect]

The compositions according to Examples 2-9 and Comparative Examples 2-5 were shaken by hand, and a visual evaluation was made regarding whether the composition showed a shiny iridescent aspect with a background of incident visible white light. The results of “YES” and “NO” are shown in Tables 2-3.

### [Sensory Tests]

Six panelists used 3 g each of the compositions according to Examples 2-9 and Comparative Examples 2-5 as a makeup remover for their own makeup. They used 3 g of the composition well shaken in their hands and massaged their dry faces with it. After massaging, they rinsed the composition by washing their face with tap water, and dried their face with tissue papers. 10 minutes later, the skin finish was evaluated by judging whether or not the panelists had a dry feeling. A dry feeling was considered as being perceived when half or more of the panelists had a dry feeling on their faces after rinsing. In this case, the result of the evaluation was indicated by “DRY”. When less than half of the panelists had a dry feeling on their face after rinsing, the result of the evaluation was indicated by “ND” (Not Dry). The results are shown in Tables 2-3.

Next, moisturized feeling was evaluated by 6 panelists after using the composition as above for 3 days. A moisturizing feeling was considered as being perceived when half or more of the panelists had moisturized feeling on the face after rinsing. In this case, the result of the evaluation was indicated by “YES”. Dry feeling was considered as being perceived when less than half of the panelists had a moisturized feeling on the face after rinsing. In this case, the result of the evaluation was indicated by “NO”. The results are shown in Tables 2-3.

TABLE 2

| | | D | RI | Ex. 2 | Ex. 3 | Ex. 4 | Ex. 5 | Ex. 6 | Ex. 7 | Ex. 8 | Ex. 9 |
|---|---|---|---|---|---|---|---|---|---|---|---|
| Oil | Ethylhexyl Palmitate | 0.85 | 1.4453 | 8.0 | 9.0 | 11.0 | 10.0 | 2.44 | 3.66 | 8.00 | 9.00 |
| | Glyeine Soybean Oil | 0.92 | 1.4740 | 29.0 | — | — | 31.0 | 7.56 | 11.34 | — | 16.35 |
| | Rapeseed Oil | 0.92 | 1.4720 | — | — | — | — | — | — | 33.7 | 16.35 |
| | Mineral Oil | 0.84 | 1.4671 | — | — | — | — | 31.0 | 26.0 | — | — |
| | Mineral Oil (longer | 0.86 | 1.4780 | — | — | 15.0 | — | — | — | — | — |
| | chain) | | | | | | | | | | |
| | Hydrogenated | 0.82 | 1.4574 | 4.0 | — | — | — | — | — | — | — |
| | Polydecene | | | | | | | | | | |
| | Shea Butter | 0.92 | 1.4730 | — | 16.0 | — | — | — | — | — | — |
| | Corn Germ Oil | 0.92 | 1.4730 | — | 16.0 | 15.0 | — | — | — | — | — |
| Polyol | Glycerin | 1.26 | 1.4720 | 12.5 | 12.5 | 12.5 | 12.5 | 12.5 | 12.5 | 14.1 | 14.1 |
| | PEG-8 | 1.13 | 1.4660 | 37.5 | 37.5 | 37.5 | 35.5 | 37.5 | 37.5 | 42.2 | 42.2 |
| Surfactant | PEG-20 Glyceryl | — | 1.4675 | 9.0 | 9.0 | 9.0 | 9.0 | 9.0 | 9.0 | 2.0 | 2.0 |
| | Triisostearate | | | | | | | | | | |

| | | | | | | | | |
|---|---|---|---|---|---|---|---|---|
| Total | 100 | 100 | 100 | 100 | 100 | 100 | 100 | 100 |
| Refractive Index of Polyol Phase | 1.4674 | 1.4674 | 1.4674 | 1.4674 | 1.4674 | 1.4674 | 1.4674 | 1.4674 |
| Refractive Index of Oil Phase | 1.4663 | 1.4665 | 1.4672 | 1.4666 | 1.4666 | 1.4666 | 1.4665 | 1.4666 |
| Δ Index | 0.0011 | 0.0009 | 0.0002 | 0.0008 | 0.0008 | 0.0008 | 0.0009 | 0.0008 |
| Aspect After Shaking | T | T | T | T | T | T | T | T |
| Shiny Iridescent Aspect | YES | YES | YES | YES | YES | YES | YES | YES |
| Skin Finish After Rinsing | ND | ND | ND | ND | ND | ND | ND | ND |
| Moisturized Feeling | YES | YES | YES | YES | YES | YES | YES | YES |

D: Density
RI: Refractive Index
T: Translucent
ND: Not Dry

TABLE 3

| | | | | Comp. | Comp. | Comp. | Comp. |
|---|---|---|---|---|---|---|---|
| | | D | RI | Ex. 2 | Ex. 3 | Ex. 4 | Ex. 5 |
| Oil | Ethylhexyl Palmitate | 0.85 | 1.4453 | — | 13.0 | 20.0 | 11.0 |
| | Mineral Oil | 0.84 | 1.4671 | 41.0 | — | — | — |
| | Mineral Oil (longer chain) | 0.86 | 1.4780 | — | 24.0 | — | 25.0 |
| | Polybutylene | 0.89 | 1.4971 | — | — | 18.0 | — |
| | Jojoba Oil | 0.86 | 1.4667 | — | 4.0 | 3.0 | — |
| | Octyldodecanol | 0.84 | 1.4540 | — | — | — | 5.0 |
| Polyol | Glycerin | 1.26 | 1.4720 | 15.0 | 12.5 | 12.5 | 12.5 |
| | PEG-8 | 1.13 | 1.4660 | 35.0 | 37.5 | 37.5 | 37.5 |
| Surfactant | PEG-20 Glyceryl Triisostearate | — | 1.4675 | 9.0 | 9.0 | 9.0 | 9.0 |

| | | | | |
|---|---|---|---|---|
| Total | 100 | 100 | 100 | 100 |
| Refractive Index of Polyol Phase | 1.4677 | 1.4674 | 1.4674 | 1.4674 |
| Refractive Index of Oil Phase | 1.4671 | 1.4665 | 1.4678 | 1.4662 |
| Δ Index | 0.0006 | 0.0009 | 0.0004 | 0.0012 |
| Aspect After Shaking | T | T | T | T |
| Shiny Iridescent Aspect | NO | NO | NO | NO |
| Skin Finish After Rinsing | Dry | Dry | Dry | Dry |
| Moisturized Feeling | NO | NO | NO | NO |

D: Density
RI: Refractive Index
T: Translucent

### Comparative Examples 6-14

### Preparation

The following compositions according to Comparative Examples 6-14 (Comp. Ex. 6-14) were respectively prepared by mixing the ingredients shown in Table 4 at room temperature, and were poured into transparent vessels with the same volume. The numerical values for the amounts of the ingredients are all based on “% by weight” as active raw materials.

The compositions according to Comparative Examples 6-14 showed the same aspect when they were left at rest. Each composition was separated into three phases, i.e., an oil phase, a surfactant phase and a polyol phase, in this order from the top to the bottom of the vessel. The sizes of the oil phase, the surfactant phase and the polyol phase in the compositions according to Comparative Examples 6-14 were almost the same as those of the compositions according to Comparative Examples 2-5. The oil phase, the surfactant phase and the polyol phases were colorless.

### [Refractive Indexes]

The refractive index of a phase composed of the ingredient(s) in Table 4 was calculated as explained above. The index of the oil phase, and the index of the polyol phase are shown in Table 4.

### [Aspect After Shaking]

The compositions according to Comparative Examples 6-14 were shaken by hand, and a visual evaluation was made regarding whether the composition was translucent or opaque. The results are shown in Table 4.

It is clear from Tables 1-3 and Table 4 that it is necessary for the difference in the refractive indexes of adjacent phases to be less than 0.0015, preferably less than 0.0014, more preferably less than 0.0013, and even more preferably less than 0.0012, in order for the composition to have a translucent appearance after the composition is shaken.

TABLE 4

| | | | | Comp. | Comp. | Comp. | Comp. | Comp. | Comp. | Comp. | Comp. | Comp. |
|---|---|---|---|---|---|---|---|---|---|---|---|---|
| | | D | RI | Ex. 6 | Ex. 7 | Ex. 8 | Ex. 9 | Ex. 10 | Ex. 13 | Ex. 12 | Ex. 13 | Ex. 14 |
| Oil | Isohexadecane | 0.78 | 1.4391 | 41.0 | — | — | — | — | — | — | — | — |
| | Hydrogenated | 0.82 | 1.4535 | — | 41.0 | — | — | — | — | — | — | — |
| | Polyisobutene | | | | | | | | | | | |
| | Ethylhexyl | 0.85 | 1.4453 | — | — | 41.0 | — | — | — | — | — | — |
| | Palmitate | | | | | | | | | | | |
| | Mineral Oil | 0.84 | 1.4780 | — | — | — | 41.0 | — | 15.2 | — | — | — |
| | Isostearyl | 0.87 | 1.4605 | — | — | — | — | 41.0 | 25.8 | — | — | — |
| | Isostearate | | | | | | | | | | | |
| | Dimethicone | 0.90 | 1.3920 | — | — | — | — | — | — | 41.0 | — | — |
| | Dicaprylyl | 0.81 | 1.4310 | — | — | — | — | — | — | — | 41.0 | — |
| | Ether | | | | | | | | | | | |
| | Octyldo- | 0.84 | 1.4540 | | | | | | | | | 41.0 |
| | decanol | | | | | | | | | | | |
| Polyol | Glycerin | 1.26 | 1.4720 | 15.0 | 15.0 | 15.0 | 15.0 | 15.0 | 15.0 | 15.0 | 15.0 | 15.0 |
| | PEG-8 | 1.13 | 1.4660 | 35.0 | 35.0 | 35.0 | 35.0 | 35.0 | 35.0 | 35.0 | 35.0 | 35.0 |
| Sur- | PEG-20 | — | 1.4675 | 9.0 | — | 9.0 | 9.0 | 9.0 | — | 9.0 | 9.0 | 9.0 |
| factant | Glyceryl | | | | | | | | | | | |
| | Triiso- | | | | | | | | | | | |
| | stearate | | | | | | | | | | | |
| | PEG-7 | — | 1.4640 | — | 9.0 | — | — | — | 9.0 | — | — | — |
| | Glyceryl | | | | | | | | | | | |
| | Cocoate | | | | | | | | | | | |

| | | | | | | | | | |
|---|---|---|---|---|---|---|---|---|---|
| Total | 100 | 100 | 100 | 100 | 100 | 100 | 100 | 100 | 100 |
| Refractive Index of Polyol Phase | 1.4678 | 1.4678 | 1.4678 | 1.4678 | 1.4678 | 1.4678 | 1.4678 | 1.4678 | 1.4678 |
| Refractive Index of Oil Phase | 1.4391 | 1.4535 | 1.4453 | 1.4780 | 1.4605 | 1.4780 | 1.3920 | 1.4310 | 1.4540 |
| Δ Index | 0.0287 | 0.0143 | 0.0225 | 0.0102 | 0.0073 | 0.0102 | 0.0758 | 0.0368 | 0.0138 |
| Aspect After Shaking | White | White | White | White | White | White | White | White | White |
| | Opaque | Opaque | Opaque | Opaque | Opaque | Opaque | Opaque | Opaque | Opaque |

D: Density
RI: Refractive Index

1. A composition, comprising:
(a) at least one oil phase including at least one triglyceride oil;
(b) at least one polyol phase including at least one polyol; and
(c) at least one surfactant phase including at least one nonionic surfactant;
wherein
at least one of the phases (a), (b) and (c) is visually distinct from the other(s), and
the amount of the triglyceride oil is 7.5% by weight or more, preferably 10.0% by weight or more, and more preferably 15.0% by weight or more, relative to the total weight of the composition.

2. The composition according to claim 1, wherein the difference in the refractive indices of the (a) oil phase and the (b) polyol phase and/or the (a) oil phase and the (c) surfactant phase is less than 0.0020, preferably less than 0.0015, and more preferably less than 0.0010.

3. The composition according to claim 1 or 2, wherein the amount of the triglyceride oil ranges 60.0% by weight or less, preferably 50.0% by weight or less, and more preferably 40.0% by weight or less, relative to the total weight of the composition.

4. The composition according to any one of claims 1 to 3, wherein the amount of the (a) oil phase ranges from 20.0 to 70.0% by weight, preferably from 25.0 to 60.0% by weight, and more preferably from 30.0 to 50.0% by weight or less, relative to the total weight of the composition.

5. The composition according to any one of claims 1 to 4, wherein the polyol is selected from the group consisting of glycerin, diglycerin, polyglycerin, ethyleneglycol, diethyleneglycol, polyethyleneglycol, propyleneglycol, dipropyleneglycol, polypropyleneglycol, butyleneglycol, pentyleneglycol, hexyleneglycol, 1,3-propanediol, and 1,5-pentanediol.

6. The composition according to any one of claims 1 to 5, wherein the amount of the (b) polyol phase ranges from 30.0 to 80.0% by weight, preferably from 40.0 to 70.0% by weight, and more preferably from 45.0 to 65.0% by weight, relative to the total weight of the composition.

7. The composition according to any one of claims 1 to 6, wherein the nonionic surfactant has an HLB value of 18.0 or less, preferably from 4.0 to 18.0, more preferably from 6.0 to 15.0, and even more preferably from 9.0 to 13.0.

8. The composition according to any one of claims 1 to 7, wherein the nonionic surfactant is selected from esters of polyols with fatty acids with a saturated or unsaturated chain containing for example from 8 to 24 carbon atoms, preferably 12 to 22 carbon atoms, and alkoxylated derivatives thereof, such as glyceryl esters of a C₈-C₂₄ fatty acid or acids and alkoxylated derivatives thereof, polyethylene glycol esters of a C₈-C₂₄ fatty acid or acids and alkoxylated derivatives thereof, sorbitol esters of a C₈-C₂₄ fatty acid or acids and alkoxylated derivatives thereof, sugar (sucrose, glucose, alkylglycose) esters of a C₈-C₂₄ fatty acid or acids and alkoxylated derivatives thereof, ethers of fatty alcohols, ethers of sugar and a C₈-C₂₄ fatty alcohol or alcohols, and mixtures thereof.

9. The composition according to any one of claims 1 to 8, wherein the nonionic surfactant is selected from the group consisting of PEG-7 glyceryl cocoate, PEG-20 methylglucoside sesquistearate, PEG-20 glyceryl tri-isostearate, PG-5 dioleate, PG-4 diisostearate, PG-10 isostearate, PEG-8 isostearate, and PEG-60 hydrogenated castor oil.

10. The composition according to any one of claims 1 to 9, wherein the amount of the (c) surfactant phase ranges from 0.1 to 20% by weight, preferably from 0.5 to 15% by weight, and more preferably from 1 to 10% by weight, relative to the total weight of the composition.

11. The composition according to any one of claims 1 to 10, wherein the triglyceride oil is selected from the group consisting of: linseed oil, camellia oil, macadamia nut oil, sunflower oil, soybean oil, arara oil, corn oil, sasanqua oil, safflower oil, grapeseed oil, sesame oil, peanut oil, wheat germ oil, cottonseed oil, alfalfa oil, poppy oil, pumpkin oil, blackcurrant oil, evening primrose oil, millet oil, barley oil, quinoa oil, rye oil, candlenut oil, passion flower oil, musk rose oil, sweet almond oil, avocado oil, castor oil, olive oil, jojoba oil, groundnut oil, rapeseed oil, coconut oil, hazelnut oil, karite butter, shea butter, palm oil, apricot seed oil and calophyllumoil.

12. The composition according to any one of claims 1 to 11, further comprising water in an amount of 2% by weight or less, preferably 1% by weight or less, and more preferably 0.5% by weight or less, relative to the total weight of the composition.

13. The composition according to any one of claims 1 to 11, which is a cosmetic composition.

14. A cosmetic process for a dry or wet keratin substance comprising the step of applying the cosmetic composition according to claim 13 to the keratin substance, with or without mixing the (a) oil phase, the (b) polyol phase and the (c) surfactant phase in the cosmetic composition before the step of applying the cosmetic composition to the keratin substance.
